(19) United States
(12) Patent Application Publication
(10) Pub. No.: US 2008/0274183 A1
(43) Pub. Date: Nov. 6, 2008

(54) Thermoplastic articles containing a medicament

(76) Inventor: Phillip Michael Cook, Kingsport, TN (US)

Correspondence Address: Eastman Chemical Company, P.O. Box 511, Kingsport, TN, 37662-5075, US

(21) Appl. No.: 11/344,826

(22) Filed: Feb. 1, 2006

Related U.S. Application Data
(60) Provisional application No. 60/650,383, filed on Feb. 4, 2005.

(51) Int. Cl. A61K 8/81; A61K 38/00; A61K 31/56; A61K 9/14; A61K 31/225

(52) U.S. Cl. 424/484; 424/487; 424/59; 514/2; 514/171; 514/547

(57) ABSTRACT

A composition suitable for making a fiber or a film includes a biocompatible primary polymer matrix which forms a continuous phase of the composition and an active agent forming a substantially discontinuous phase of the composition. The biocompatible primary polymeric matrix has a glass transition temperature of less than about 100° C. and is impermeable to the active agent. A process for making an article, such as a fiber or a film, includes the steps of providing a polymer blend comprising a polymer matrix having from 99.9 weight % to about 30 weight % of a biocompatible polymeric matrix and from about 0.1 to about 70 weight % of an active agent, wherein the weight % are based on the total weight of the of the polymeric matrix and active agent, and extruding the polymer blend into the article.

### CROSS REFERENCE TO RELATED APPLICATIONS

Benefit is claimed to the earlier filed application having U.S. Ser. No. 60/650,383 filed Feb. 4, 2005, the entire disclosure of which is incorporated herein by reference.

### BACKGROUND OF THE INVENTION

The present invention relates to a polymeric article having an active agent or medicament releasably incorporated therein. More particularly, the present invention relates to an extruded polymeric fiber having a medicament releasably incorporated therein, a method for making a medicated fiber and articles made therefrom. Advantageously, the present invention enables an active medicament or agent to be gradually released from the fiber through contact interaction of a solvent on the active agent or medicament on and in the fiber.

In administering a medicament, one objective is to have a fairly constant concentration of the active agent released over time so that spikes in medication concentration in blood serum are reduced or eliminated. One method for administering medications is to incorporate a medicament into a carrier that releases the medicament over time. The inclusion of active materials on or in polymeric articles is well known and described in the literature. Such articles are made into various shapes and dimensions, such as granules, films, fibers, containers, and structural components. For example, U.S. Pat. No. 4,713,291 issued to Sasaki et al. on Dec. 15, 1987 discloses a thermoplastic polymer fiber in a hollow sheath-core type of configuration. The hollow core contains a perfume dispersed in the interior of the polymer. Suitable polymers for the core include polyethylene homopolymers and copolymers. Suitable polymers for the sheath include polyolefins, polyamides and polyesters.

U.S. Pat. No. 4,764,377 issued to Goodson on Aug. 16, 1988 discloses a method for the treatment of periodontal disease by delivering a therapeutic agent by packing the gingival crevice with a thermoplastic fiber that has been impregnated with the therapeutic agent. The thermoplastic fiber is permeable to the therapeutic agent. Suitable thermoplastic materials are collagen, glycolic acid polymers, methacrylate polymers and polylactides.

U.S. Pat. No. 5,180,585 issued to Jacobson et al. on Jan. 19, 1993 discloses a polymeric particle having antimicrobial properties. The polymeric particle has a core material that is coated with a metal compound selected from silver, silver oxide, silver halides, copper, copper (I) oxide, copper (II) oxide, copper sulfide, zinc oxide, zinc sulfide, and zinc silicate. The first coating is then coated with a protective layer selected from silica, silicates, borosilicates, aluminosilicates, alumina, and aluminum phosphate. The particle can be incorporated into paints, coatings, caulk, grouts, mortar, cement and masonry products or such shaped articles as films and fibers.

U.S. Pat. No. 6,517,759 issued to Ferenc et al. on Feb. 11, 2003 discloses a fragrance emitting fiber that may include a releasable antimicrobial agent.

U.S. Pat. No. 6,685,957 issued to Bezemer et al. on Feb. 3, 2004 discloses a fibrous polymer implant for the controlled release of a bioactive agent in vivo. The fiber is prepared by wet spinning an aqueous solution added to a solution of amphiphilic block copolymer containing hydrophilic blocks, such as polyalkylene glycol and hydrophobic blocks such as an aromatic ester dissolved in a first solvent immiscible with water to form an emulsion. The emulsion is injected through a nozzle into a second solvent miscible with the first solvent in which the copolymer is essentially insoluble to form a solid copolymer fiber loaded with the bioactive agent.

U.S. Pat. No. 4,978,537 issued to Song on Dec. 18, 1990 discloses a chewing gum which has a gradual release structure. The gradual release structure is formed by melt spinning a mixture of active agent and wall material into a fiber. The gradual release structure is made by preparing a mixture of active agent and wall material, having more than zero but less than about 55 percent by weight active agent. This mixture is melt spun into a fiber which is cut. The gradual release structure, a gum base, and a water soluble bulk portion are combined to form the chewing gum.

R. Dunn & D. Lewis, “Fibrous Polymers for the Delivery of Contraceptive Steroids to the Female Reproductive Tract”, Controlled Release of Pesticides and Pharmaceuticals 125-46 (D. Lewis ed. 1981), discloses the application of a controlled release of a contraceptive active agent by incorporating the active agent in a polymer fiber. The article discloses two types of diffusion-controlled systems: 1) a monolithic fiber/active agent systems where an active agent is combined with a polymer in a manner where the agent is dissolved or dispersed throughout the polymer matrix; and 2) hollow core-shell fibers where the active agent resides inside the hollow fiber. The article teaches that polyamides, polyethylene, polypropylene, polycaprolactone, and poly (DL-lactide) are suitable polymers. A problem recognized by the authors was that temperature necessary for melt extrusion of the polymer caused degradation of the active agent.

Accordingly, there is a need for a melt-extrudable, polymeric fiber that is suitable for food grade applications, and will permit internal administration wherein the active ingredient is incorporated into the fiber matrix and is gradually released.

### SUMMARY OF THE INVENTION

Briefly, in one embodiment, the present invention provides a polymeric article, such as a film or fiber, having a gradual release of an active agent. The polymeric article is suitable for food grade applications, and can be incorporated with or formed into a variety of solid or liquid consumables, such as pills, caplets, cough syrups, protein drinks or added to a variety of food products. The composition comprises a biocompatible primary polymeric matrix and an active agent, which desirably is suitable for pharmaceutical or biological purposes. The biocompatible primary polymeric matrix has a glass transition temperature of less than about 100° C., and is impermeable to the active agent.

Another aspect of the present invention is a process for making the polymeric composition by providing a blend of from 99.9 weight % to about 30 weight % of a biocompatible polymeric matrix and from about 0.1 to about 70 weight % of the bioactive agent, and extruding or casting the polymer blend into a fiber having a denier of from about 1 to about 5000 or a film having a thickness of from about 0.001 to about 10.0 mm in thickness.

Advantages of the present invention will become apparent to those skilled in the art in view of the following description and the accompanying drawings wherein like parts and objects in the several views have similar reference numerals. It is to be understood that the inventive concept is not to be considered limited to the constructions disclosed herein but instead by the scope of the appended claims.

### BRIEF DESCRIPTION OF THE DRAWINGS

FIG. 1 is an illustration of a polymeric article (fiber) of the present invention showing an active agent as areas in a discontinuous phase.

FIG. 2 is an illustration of a polymeric article (fiber) of the present invention showing the gradual release of the active agent after it has been exposed to a solvent for a period of time sufficient to dissolve a portion of the active agent.

FIG. 3 is a scanning electron photomicrograph at 1000 magnification showing the surface interstitial opening of a water-extracted glycerol/cellulose diacetate article (film) of the present invention having a 1:1 weight ratio of cellulose diacetate to glycerol.

FIG. 4 is a scanning electron photomicrograph at 5000 magnification of the article of FIG. 3.

FIG. 5 is a scanning electron photomicrograph at 1000 magnification showing the surface interstitial opening of a water-extracted glycerol/cellulose diacetate article (film) having a 1:3 weight ratio of cellulose diacetate to glycerol.

FIG. 6 is a scanning electron photomicrograph at 1000 magnification showing the surface interstitial opening of a water-extracted glycerol/cellulose diacetate article (film) having a 1:2 weight ratio of cellulose diacetate to glycerol.

FIG. 7 is a scanning electron photomicrograph at 1000 magnification showing the surface interstitial openings of a water-extracted cellulose diacetate article (film) containing powdered sugar in a 1:1 weight ratio of cellulose diacetate to sugar.

FIG. 8 is a scanning electron photomicrograph at 1000 magnification showing the surface interstitial openings of a water-extracted cellulose diacetate article (film) containing powdered sugar in a 1:2 weight ratio of cellulose diacetate to sugar.

### DETAILED DESCRIPTION OF THE INVENTION

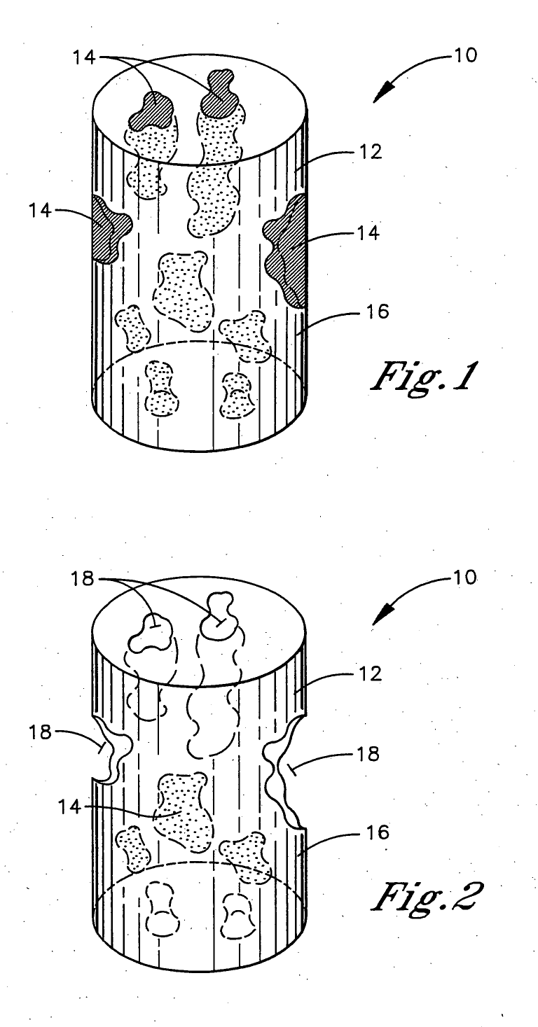

Referring to FIGS. 1 and 2, an article 10 for the gradual release of an active agent is illustrated as a doped or loaded fiber or film composed of a biocompatible polymeric matrix 12 forming a substantially continuous phase and an active agent 14 forming a substantially discontinuous or dispersed phase. As used herein, the term “continuous phase” means that the polymer matrix 12 forms a substantially unitary compositional matrix, notwithstanding the existence of pockets of active agent intermediate identifiable portions of the polymer matrix, which continue after interruption. As used herein, the term “discontinuous phase” or “dispersed phase” means discrete identifiable pockets, segments or areas that are isolated by, dispersed in or defined by the continuous phase, which may, depending upon the amount or concentration of the active agent, be interconnected with adjacent identifiable pockets, segments or areas of the active agent. For the sake of brevity and clarity, the polymeric article will hereinafter be described in terms of a fiber. The polymer matrix 12 is impermeable to the active agent 14 and desirably is substantially insoluble in the targeted solvent for the active agent. In preparing the doped or loaded polymeric fiber, a mixture of the polymer matrix and active agent, is prepared by melt spinning the combination into a filament of a predetermined width. The filament is then cut to the desired length to make the loaded fiber. As used herein the terms “doped”, “loaded”, and “impregnated” are used interchangeably to convey the general understanding that the active agent is admixed into the polymer and not reacted with the polymer. Desirably, the active agent 14 is homogeneously distributed throughout the polymeric matrix 12 of the fiber 10, although it is possible for the active agent 14 is present as random pockets where the active agent is at a substantially higher concentration than an adjacent portion of the polymer matrix in the fiber 10. A plurality of doped polymeric fibers may be compressed into a solid delivery device such as pill, caplet or other administrative delivery device known to those skilled in the art. Alternatively, the loaded fiber may be admixed with a liquid or syrup for oral or subcutaneous injection. As used herein, the term “homogeneously distributed” means that in a random sample of approximately 0.5 gram of the loaded fiber and a sample size of “n”, where “n” represents the number of samples, the mean difference in the amount of active agent in the samples will be no more than about 25 weight %, when “n” is greater than 10. As one skilled in the art will understand by the description which follows, the amount of active agent present in each individual chopped fiber may vary, but a sample having a statically significant number of individual fibers will be substantially uniform in the amount of active agent present.

The active agent 14 is dispersed throughout the polymer matrix 12 and may be in contact with itself forming contiguous pockets within the polymer matrix. The active agent 14, however, does not necessarily have to be in a contiguous phase. The ends of the doped polymeric fiber 10 may have openings, exposing the active agent. Additionally, the active agent may be exposed along the sides 16 of the fiber 10. Advantageously, the active agent is gradually released through direct interaction with a solvent, such as saliva, gastric enzymes, lymphatic fluids, and the like.

The polymer matrix may be any kind of polymer that is biocompatible. This permits the use of a polymer that can be loaded, doped, or impregnated with the bioactive agent for pharmaceutical and/or biological purposes. In the context of the present invention, the term “biocompatible” is intended to refer to materials which may be incorporated into a human or animal body substantially without unacceptable responses from the human or animal, or which does not elicit an immune response or allergic reaction. Preferably, the polymer matrix comprises from about 5 to 100 weight % of a primary polymer comprising a cellulosic material selected from the group consisting of cellulose acetate, cellulose acetate propionate, and cellulose acetate butyrate available from Eastman Chemical Company, Kingsport, Tenn.

The loaded polymer fiber comprises from about 99.9 weight % to about 30 weight % of a biocompatible polymeric matrix and from about 0.1 to about 70 weight % of an active agent, preferably from about 55 weight % to about 99 weight % of a biocompatible polymeric matrix and from about 45 weight % to about 1 weight % of an active agent, more preferably from about 65 weight % to about 99 weight % of a biocompatible polymeric matrix and from about 35 weight % to about 1 weight % of an active agent, and most preferably from about 75 weight % to about 99 weight % of a biocompatible polymeric matrix and from about 25 weight % to about 1 weight % of an active agent, wherein the above weight percentages are based on the total weight of the polymeric matrix and active agent.

It is to be understood that the ranges explicitly recited herein further includes all ranges that would be implicitly included therein. For example, an explicit range of 55 to 99 weight % would implicitly include the ranges 56 to 99 weight %, 57 to 99 weight % and the like, as well as ranges 56 to 98 weight %, 56 to 97 weight %, and the like.

The polymer matrix may also contain a secondary biocompatible polymer. When a secondary polymer is included into the polymer matrix, desirably the secondary polymer has a melt temperature that is less than the temperature at which the active agent thermally decomposes or substantially decreases in bioactivity. Although not necessary, it is preferred that the secondary polymer be compatible with the primary polymer so as to not incur a phase separation between the primary and secondary polymers. Non-limiting examples of suitable secondary polymers include biocompatible natural or synthetic polymers, such as collagen, glycolic acid polymers, methacrylate polymers, ethylene vinyl acetate polymers, ethylene vinyl alcohol copolymers, polycaprolactone, and polylactide polymers. The secondary polymer can be: biodegradable, at least partially permeable to the active agent, soluble in the solvent, or any combination thereof.

Desirably, when the polymer matrix includes a second biocompatible polymer, the polymer matrix comprises from about 5 weight % to 100 weight % of the primary polymer and up to about 95 weight % of the secondary polymer, based on the total weight of the polymer matrix. Preferably, the when the polymer matrix includes a secondary biocompatible polymer, the polymer matrix comprises from about 25 weight % to 90 weight % of the primary polymer and from about 75 weight % to about 10 weight % of the secondary polymer, based on the total amount of polymer in the matrix, and more preferably, polymer matrix has greater than about 50 weight % of the primary polymer with the remainder of the polymer matrix being composed of the secondary biocompatible polymer.

The active agent(s) which is/are to be loaded into the polymer may be chosen from various groups of compounds and desirably includes one or more of a biologically active agent. The term “biologically active agent” or “bioactive agent”, as used herein, includes an agent which provides a therapeutic or prophylactic effect, a compound that may be able to invoke a biological action such as an immune response, or could play any other role in one or more biological processes such as a compound that affects or participates in tissue growth, cell growth, cell differentiation. Such agents include, but are not limited to, immunosuppressants, water and oil soluble vitamins, antioxidants, anesthetics, chemotherapeutic agents, steroids (including retinoids), hormones, antibiotics, anti-virals, anti-fungals, anti-tumor agents antiproliferatives, antihistamines, anticoagulants, antiphotoaging agents, melanotropic peptides, nonsteroidal and steroidal anti-inflammatory compounds, antipsychotics, and radiation absorbers, including UV-absorbers, lipids, lipopolysaccharides, and peptides, polypeptides and proteins in general.

More specifically, non-limiting examples of such biologically active agent(s) include the following:

1. Growth factors such as Bone Morphogenetic Proteins (BMP), epidermal growth factors, e.g. Epidermal Growth Factor (EGF), fibroblast growth factors, e.g. basic Fibroblast Growth Factor (bFGF), Nerve Growth Factor (NGF), Bone Derived Growth Factor (BDGF), transforming growth factors, e.g. Transforming Growth Factor-.beta.1 (TGF-.beta.1), and human Growth Hormone (hGH);

2. Viral surface antigens or parts of viruses: adenoviruses, Epstein-Barr Virus, Hepatitis A Virus, Hepatitis B Virus, Herpes viruses, HIV-1, HIV-2, HTLV-III, Influenza viruses, Japanese encephalitis virus, Measles virus, Papilloma viruses, Paramyxoviruses, Polio Virus, Rabies, Virus, Rubella Virus, Vaccinia (Smallpox) viruses, and Yellow Fever Virus;

3. Bacterial surface antigens or parts of bacteria: Bordetella pertussis, Helicobacter pylorn, Clostridium tetani, Corynebacterium diphtheria, Escherichia coli, Haemophilus influenza, Klebsiella species, Legionella pneumophila, Mycobacterium bovis, Mycobacterium leprae, Mycrobacterium tuberculosis, Neisseria gonorrhoeae, Neisseria meningitidis, Proteus species, Pseudomonas aeruginosa, Salmonella species, Shigella species, Staphylococcus aureus, Streptococcus pyogenes, Vibrio cholera, Yersinia pestis;

4. Surface antigens of parasites causing disease or portions of parasites: Plasmodium vivax—malaria, Plasmodium falciparum—malaria, Plasmodium ovale—malaria, Plasmodium malariae—malaria, Leishmania tropica—leishmaniasis, Leishmania donovani, leishmaniasis, Leishmania branziliensis—leishmaniasis, Trypanosoma rhodescense—sleeping sickness, Trypanosoma gambiense—sleeping sickness, Trypanosoma cruzi—Chagas' disease, Schistosoma mansoni—schistosomiasis, Schistosomoma haematobium—schistomiasis, Schistosoma japonicum—shichtomiasis, Trichinella spiralis—trichinosis, Stronglyloides duodenale—hookworm, Ancyclostoma duodenale—hookworm, Necator americanus—hookworm, Wucheria bancrofti—filariasis, Brugia malaya—filariasis, Loa loa—filariasis, Dipetalonema perstaris—filariasis, Dracuncula medinensis—filariasis, and Onchocerca volvulus—filariasis;

5. Immunoglobulins: IgG, IgA, IgM, Antirabies immunoglobulin, and Antivaccinia immunoglobulin;

6. Antitoxins: Botulinum antitoxin, diphtheria antitoxin, gas gangrene antitoxin, and tetanus antitoxin;

7. Antigens which elicit an immune response against: Foot and Mouth Disease, hormones and growth factors such as follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone; cytokines, such as interferons, interleukins, colony stimulating factors, and tumor necrosis factors: fibrinolytic enzymes, such as urokinase, kidney plasminogen activator; and clotting factors, such as Protein C, Factor VIII, Factor IX, Factor VII and Antithrombin III;

8. Other proteins or peptides selected from: albumin, atrial natriuretic factor, renin, superoxide dismutase, α₁-antitrypsin, lung surfactant proteins, bacitracin, bestatin, cydosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-.alpha.-endorphin, gonadotropin releasing hormone, leuprolide, α-MSH, and metkephamid;

9. Anti-tumor agents: altretamin, fluorouracil, amsacrin, hydroxycarbamide, asparaginase, ifosfamid, bleomycin, lomustin, busulfan, melphalan, chlorambucil, mercaptopurin, chlormethin, methotrexate, cisplatin, mitomycin, cyclophosphamide, procarbazin, cytarabin, teniposid, dacarbazin, thiotepa, dactinomycin, tioguanin, daunorubicin, treosulphan, doxorubicin, tiophosphamide, estramucin, vinblastine, etoglucide, vincristine, etoposid, and vindesin;

10. Penicillins: ampicillin, nafcillin, amoxicillin, oxacillin, azlocillin, penicillin G, carbenicillin, penicillin V, dicloxacillin, phenethicillin, floxacillin, piperacillin, mecillinam, sulbenicillin, methicillin, ticarcillin, and mezlocillin;

11. Cephalosporins: cefaclor, cephalothin, cefadroxil, cephapirin, cefamandole, cephradine, cefatrizine, cefsulodine, cefazolin, ceftazidim, ceforanide, ceftriaxon, cefoxitin, cefuroxime, cephacetrile, latamoxef, and cephalexin;

12. Aminoglycosides: amikacin, neomycin, dibekacyn, kanamycin, gentamycin, netilmycin, kanamycin, and tobramycin;

13. Macrolides: amphotericin B, novobiocin, bacitracin, nystatin, clindamycin, polymyxins, colistin, rovamycin, erythromycin, spectinomycin, lincomycin, and vancomycin;

14. Tetracyclines: chlortetracycline, oxytetracycline, demeclocycline, rolitetracycline, doxycycline, tetracycline, and minocycline;

15. Sulfonamides: sulfadiazine, sulfamethizol, sulfadimethoxin, sulfamethoxazole, sulfadimidin, sulfamethoxypyridazine, sulfafurazole, sulfaphenazol, sulfalene, sulfisomidin, sulfamerazine, sulfisoxazole, and trimethoprim with sulfamethoxazole or sulfametrole;

16. Urinary tract antiseptics: methanamine, quinolones (norfloxacin, cinoxacin), nalidixic acid, nitro-compounds (nitrofurantoine, nifurtoinol), and oxolinic acid;

17. Drugs for tuberculosis: aminosalicyclic acid, isoniazide, cycloserine, rifampicine, ethambutol, tiocarlide, ethionamide, and viomycin;

18. Drugs for leprosy: amithiozone, rifampicine, clofazimine, and sodium sulfoxone, diaminodiphenylsulfone (DDS, dapsone);

19. Antifungal agents: amphotericin B, ketoconazole, clotrimazole, miconazole, econazole, natamycin, flucytosine, nystatine, and griseofulvin;

20. Antiviral agents: aciclovir, idoxuridine, amantidine, methisazone, cytarabine, vidarabine, and ganciclovir;

21. Chemotherapy of amebiasis: chloroquine, iodoquinol, clioquinol, metronidazole, dehydroemetine, paromomycin, diloxanide, furoatetinidazole, and emetine;

22. Anti-malarial agents: chloroquine, pyrimethamine, hydroxychloroquine, quinine, mefloquine, sulfadoxine/pyrimethamine, pentamidine, sodium suramin, primaquine, trimethoprim, and proguanil;

23. Anti-helmninthiasis agents: antimony potassium tartrate, niridazole, antimony sodium dimercaptosuccinate, oxamniquine, bephenium, piperazine, dichlorophen, praziquantel, diethylcarbamazine, pyrantel parmoate, hycanthone, pyrivium pamoate, levamisole, stibophen, mebendazole, tetramisole, metrifonate, thiobendazole, and niclosamide;

24. Anti-inflammatory agents: acetylsalicyclic acid, mefenamic acid, aclofenac, naproxen, azopropanone, niflumic acid, benzydamine, oxyphenbutazone, diclofenac, piroxicam, fenoprofen, pirprofen, flurbiprofen, sodium salicyclate, ibuprofensulindac, indomethacin, tiaprofenic acid, ketoprofen, and tolmetin;

25. Anti-gout agents: colchicine, and allopurinol;

26. Centrally acting (opoid) analgesics: alfentanil, methadone, bezitramide, morphine, buprenorfine, nicomorphine, butorfanol, pentazocine, codeine, pethidine, dextromoramide, piritranide, dextropropoxyphene, sufentanil, and fentanyl;

27. Local anesthetics: articaine, mepivacaine, bupivacaine, prilocalne, etidocaine, procaine, lidocaine, and tetracaine;

28. Drugs for Parkinson's disease: amantidine, diphenhydramine, apomorphine, ethopropazine, benztropine mesylate, lergotril, biperiden, levodopa, bromocriptine, lisuride, carbidopa, metixen, chlorphenoxamine, orphenadrine, cycrimine, procyclidine, dexetimide, and trihexyphenidyl;

29. Centrally active muscle relaxants: baclofen, carisoprodol, chlormezanone, chlorzoxazone, cyclobenzaprine, dantrolene, diazepam, febarbamate, mefenoxalone, mephenesin, metoxalone, methocarbamol, and tolperisone;

30. Corticosteroids and Mineralocorticosteroids: cortisol, desoxycorticosterone, and fluorohydrocortisone;

31. Glucocorticosteroids: beclomethasone, betamethasone, cortisone, dexamethasone, fluocinolone, fluocinonide, fluocortolone, fluorometholone, fluprednisolone, flurandrenolide, halcinonide, hydrocortisone, medrysone, methylprednisolone, paramethasone, prednisolone, prednisone, and triamcinolone (acetonide);

32. Androgenic steroids used in therapy: danazole, fluoxymesterone, mesterolone, methyltestosterone, testosterone and salts thereof;

33. Anabolic steroids used it therapy: calusterone, nandrolone and salts thereof, dromostanolone, oxandrolone, ethylestrenol, oxymetholone, methandriol, stanozolol methandrostenolone, and testolactone;

34. Antiandrogens: cyproterone acetate;

35. Estrogenic steroids used in therapy: diethylstilbestrol, estradiol, estriol, ethinylestradiol, mestranol, and quinestrol;

36. Anti-estrogens: chlorotrianisene, clomiphene, ethamoxytriphetol, nafoxidine, and tamoxifen;

37. Allylestrenol, desogestrel, dimethisterone, dydrogesterone, ethinylestrenol, ethisterone, ethynadiol diacetate, etynodiol, hydroxyprogesterone, levonorgestrel, lynestrenol, medroxyprogesterone, megestrol acetate, norethindrone, norethisterone, norethynodrel, norgestrel, and progesterone.

38. Thyroid drugs used in therapy: levothyronine and liothyronine; and

39. Anti-thyroid drugs used in therapy: carbimazole, methimazole, methylthiouracil, and propylthiouracil.

Depending on the type of polymer and the nature of the active agent(s), the amount of active agent incorporated in and on the fiber may vary. Generally, the total amount of active agent (excluding any preservatives, antioxidants or solubilizing agents) admixed into the loaded fiber is from about 0.01 weight % to about 70 weight %, based on the total weight of the polymeric matrix and active agent in the loaded fiber. Desirably, the amount of active agent admixed into the loaded fiber is from about 1.0 to about 35 weight %, and more preferably is from about 1.0 to about 25 weight %, and most preferably is from about 1.0 to about 10 weight %, based on the total weight of the polymeric matrix and active agent in the loaded fiber.

When a hydrophobic drug is incorporated into the polymer fiber, preferably at least one hydrophobic antioxidant is present. Such hydrophobic antioxidants retard the release of the biologically active agent and may retard the degradation of the copolymer, if present. The hydrophobic antioxidant(s) may be present in the loaded fiber in an amount of from about 0.1 weight % to about 10 weight % of the total weight of the active agent, and preferably is from about 0.5 weight % to about 2 weight %, based on the total weight of the active agent.

When a hydrophobic active agent or drug is incorporated into the polymer fiber, a water-soluble preparation of a fat-soluble vitamin can also be included to improve or enhance the solubility of the hydrophobic active agent. Desirably, the solubility enhancing preparation is admixed with the active agent prior to the active agent being added to the polymer matrix. Preferably, the water-soluble preparation of a fat-soluble vitamin is a water soluble derivative of well known vitamin E-active tocopherols, such as those disclosed in U.S. Pat. No. 2,680,749, the entire disclosure of which is incorporated herein by reference. Generally, the water-soluble tocopherol derivatives are prepared by esterifying any tocopheryl acid ester with polyethylene glycol. The polyoxyethylene glycol moiety has a molecular weight in the range of about 200 to 20,000, preferably of about 400 to about 10,000, more preferably from about 400 to about 1000 and most preferably the water-soluble preparation of a fat-soluble vitamin is vitamin E polyethylene glycol 1000 succinate available from Eastman Chemical Company under the trade name Vitamin E 1000 TPGS™. The commercial product is prepared by esterifying the carboxyl group of crystalline d-α-tocopheryl acid succinate (or the d,l-form in the case of synthetic vitamin E) with polyethylene glycol 1000. Vitamin E 1000 TPGS™ is very stable and does not hydrolyze under normal conditions. It is essentially tasteless and odorless and has been shown to be a readily bioavailable source of vitamin E for individuals or animals having difficulties absorbing naturally occurring, fat-soluble vitamin E. Its therapeutic benefit has been well documented and recognized. The amount of water-soluble tocopherol derivative incorporated into the doped fiber is from about 0.02 to about 10 weight %, preferably, from about 0.1 to about 5 weight %, and more preferably from about 0.1 to about 3 weight %, based on the total weight of the active agent and water-soluble tocopherol. Desirably, the vitamin E succinate polyethylene glycol 1000 is dissolved in a solvent compatible with the active agent and is blended with the active agent prior to being incorporated with the polymer matrix.

In accordance with another aspect of the present invention, the loaded polymeric fiber is prepared by providing a polymer blend comprising a polymer matrix having from 99.9 weight % to about 30 weight % of a biocompatible polymeric matrix and from about 0.1 to about 70 weight % of an active agent, based on the total weight of polymer matrix and active agent; and extruding or wet spinning the polymer blend into a fiber having a denier of from about 1 to about 5000. When melt spun, to prevent the thermal degradation of the active agent, the biocompatible polymeric matrix should have a glass transition temperature of less than about 100° C. Desirably, the biocompatible polymeric matrix is impermeable to the active agent. In a preferred embodiment, the polymer blend comprises from about 55 weight % to about 99 weight % of a biocompatible polymeric matrix and from about 45 weight % to about 1 weight % of an active agent, more preferably from about 65 weight % to about 99 weight % of a biocompatible polymeric matrix and from about 35 weight % to about 1 weight % of an active agent, and most preferably from about 75 weight % to about 99 weight % of a biocompatible polymeric matrix and from about 25 weight % to about 1 weight % of an active agent, wherein the above weight percentages are based on the total weight of the polymer matrix and active agent. The polymer blend is extruded into a fiber using extrusion techniques well known to those skilled in the polymer arts. For example, the fibers can be prepared using melt blown or spunbond techniques.

In one aspect of the invention, the loaded fibers are prepared by blending predetermined amounts of the polymer matrix and the active agent in a mixer until substantially homogeneous. The mixture, which may be a dry powder, granules or pellets, melted or a mixture of dry and melted polymer, is then transferred to an extruder. The mixture is melted and extruded through orifices or spinneret of a heated nozzle into a stream of a hot gas. The temperature of the hot gas is typically greater than ambient temperature but less than the die temperature. Desirably, the fibers have a basis weight of about 0.1 denier to about 5000 denier, preferably from about 3 denier to about 500 denier and more preferably from about 10 denier to about 100 denier. A denier is a unit of weight indicating the fineness of a fiber filament and is equal to a weight of fiber, in grams, per 9000 meters.

The loaded fibers, once extruded, are cut into a length suitable for the desired use desirably, the fibers are cut to length of from about 0.01 millimeter (mm) to about 5 mm and more preferably are from about 0.1 mm to about 3 mm.

Alternatively, the loaded fibers can be prepared using a wet-spinning technique known to those skilled in the art. Briefly, wet-spinning involves solubilizing the fiber matrix and active agent in a common solvent or in separate solvents, combining the two solubilized materials to form a solubilized blend, and forcing or extruding the solubilized blend through a spinneret of a predetermined sized to form a fiber. Generally, the solvent(s) is (are) removed from the fiber through evaporation using techniques known to those skilled in the art. Optionally, when forming the porous fiber of the present invention using a wet-spinning technique, a morphology promoting agent is included or added prior to spinning or extruding to assist in forming the appropriate sized voids in the resultant fiber. Preferably, the morphology promoting agent is water-soluble and includes such materials as powdered sugar, glycerol, sodium chloride, and potassium chloride. The amount of morphology promoting agent added to the solubilized blend is from about 1:1 to about 5:1 morphology promoting agent to fiber matrix, and preferably is from about 1:1 to about 2:1 of morphology promoting agent to fiber matrix, wherein the above ratios are on a dry weight basis. The morphology promoting agent may be removed (dissolved) prior to further processing of the fiber or may remain in the fiber and dissolved upon ingestion.

Referring again to FIGS. 1 and 2, although not to be bound by any theory, it is believed that gradual release of the active agent 14 occurs when the fiber is brought into contact with a solvent or otherwise dispersing media for the active agent. The solvent first dissolves the active agent 14 in the sides and openings at the ends of the fiber 10. If the active agent is in a contiguous phase within the polymer matrix, the active agent in those openings is dissolved and released, and spaces or channels 18 in the support matrix are created. The solvent fills these channels 18 and begins to dissolve the newly exposed active agent, which was in contact with the now dissolved active agent located in the openings at ends of the support matrix. Thus, the length of the channels 18 in the support matrix gradually increases as the active agent directly in contact with the solvent is dissolved. The polymeric matrix composing the fiber and which defines the interstitial openings within the fiber is less soluble in the solvent than the active agent. Preferably the wall material is substantially insoluble in the solvent under the conditions in which the fiber is being used. As used herein the term “substantially insoluble” means that less than about 5 parts of the polymer matrix would dissolve in 100 parts of solvent at room temperature and preferably less than about 2 parts of the polymer matrix would dissolve in 100 parts of solvent at room temperature.

It is presently believed that the polymer matrix does not prevent the dissolution of the active agent. Instead it is believed that the support matrix serves to limit the rate of dissolution by restricting the area of active agent in direct contact with the solvent to the ends of the channels within the support matrix. Thus, the solvent gradually works its way into the fiber, dissolves or disperses the active agent then slowly diffuses back to the outer surface for absorption. When a secondary polymer is included in the matrix, and the secondary polymer is soluble in the solvent, it is believed that a greater amount of active agent included in the fiber will be available for utilization.

The loaded fibers of the present invention may be utilized in making a wide variety of products. For example, the loaded fibers can be incorporated into a tablet, caplet, or other solid dosage for pharmaceutical applications. Such tablet or caplet can be coated or encapsulated using techniques known to those skilled in the art. The tablet can be coated using compositions that have high organic solubility, good film-forming properties and low water solubility give better delayed release, while compositions that have high water solubility give faster release. Such low water-solubility compositions include acrylic polymers and copolymers, carboxyvinyl polymer, polyamides, polystyrene, polyvinyl acetate, polyvinyl acetate phthalate, polyvinylpyrrolidone and waxes. Although all of these materials are possible for encapsulation of the loaded fiber, only food-grade materials should be considered such as materials like agar, alginates, a wide range of cellulose derivatives like ethyl cellulose, methyl cellulose, sodium hydroxymethyl cellulose, and hydroxypropylmethyl cellulose, dextrin, gelatin, and modified starches.

The amount of coating or encapsulating material on the tablet may also control the length of time before the solvent effectively contacts the loaded fiber and releases the medicament from the fiber. The release rate is generally not instantaneous, but gradual over an extended period of time. Generally, the coating will be about 5 to about 20 weight % based on the total weight of the coated tablet and preferably, the coating will be about 5 to about 10 weight %. Depending on the coating material, a higher or lower amount of coating material may be needed to give the desired rate of release.

Alternatively, the loaded fibers of the present invention can be incorporated into a variety of food grade products, such as cereals, breads, food supplement bars such as energy bars and snack bars, crackers, muffins, cookies, candies, chips and soft drinks. It is also within the scope of the present invention to suspend the fibers in nutritional beverages having appropriate viscosity, soups, puddings, and mousses. These fibers could also be added to a variety of food supplement preparations consisting of vitamins, minerals, and nutrients in the form of tablets and capsules.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### EXAMPLES 1-3

To investigate the utility of nutrient release from the polymeric articles, films were used as a model for screening completeness of delivery and for achieving the desired morphology likely to be needed to maximize release of active agents. Although, not to be bound by any theory, it is believed that a doped or loaded fiber in accordance with the present invention would behave in a similar manner. Films were made from solutions containing 10-20% cellulose diacetate and 80-90% by weight of acetone. If preparing a fiber, a solution containing 20-30% cellulose diacetate and 70-80% acetone by weight may be used. The cellulose diacetate used is commercially available from Eastman Chemical Company and is known as CA 394-60. Its acetyl content is 40.0±0.5 weight %. Using test method ASTM-A, the cellulose acetate had a viscosity of 24-44, using test method ASTM Method D 1343 its viscosity was measured at 60 seconds. Its glass transition temperature of the cellulose acetate was 180° C.

In a vial, an amount of glycerol was added to a 10.0.0 gram aliquot of 10.0% cellulose diacetate in acetone to bring the mixture to the specified cellulose diacetate:glycerol ratio shown in Table I below. The vial was sealed and then agitated until the sample was homogeneous. A 20 micron film was drawn down on a glass plate and set aside to dry. The film was allowed to dry overnight. A piece of the film was then removed, weighed, and placed in another vial containing a sodium phosphate/sodium hydroxide solution buffered to a pH of 7.3. The vial was gently agitated for 24 hours at a temperature of about 25° C., after which time it was removed from the liquid, dried, and weighed. The extraction procedure was repeated once more. The extraction data are given in Table I below.

TABLE I

| | Example # | | |
|---|---|---|---|
| | 1 | 2 | 3 |
| Ratio CE:glycerol | 1:1 | 1:2 | 1:3 |
| Dry film wt, initial | 0.397 g | 0.575 g | 0.659 g |
| Dry film wt, after 1 extraction | 0.122 g | 0.108 g | 0.099 g |
| Dry film wt, after 2 extractions | 0.118 g | 0.074 g | 0.099 g |

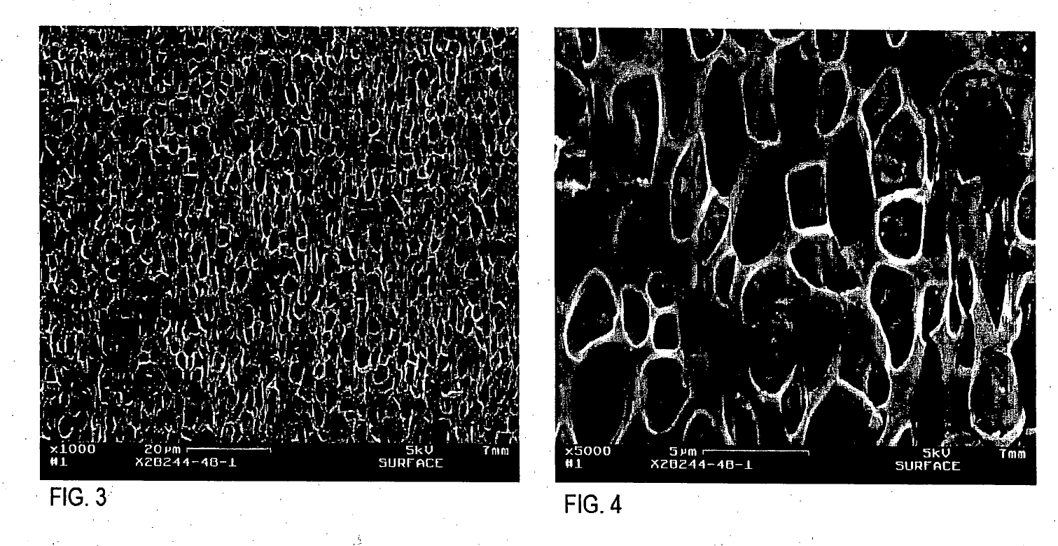

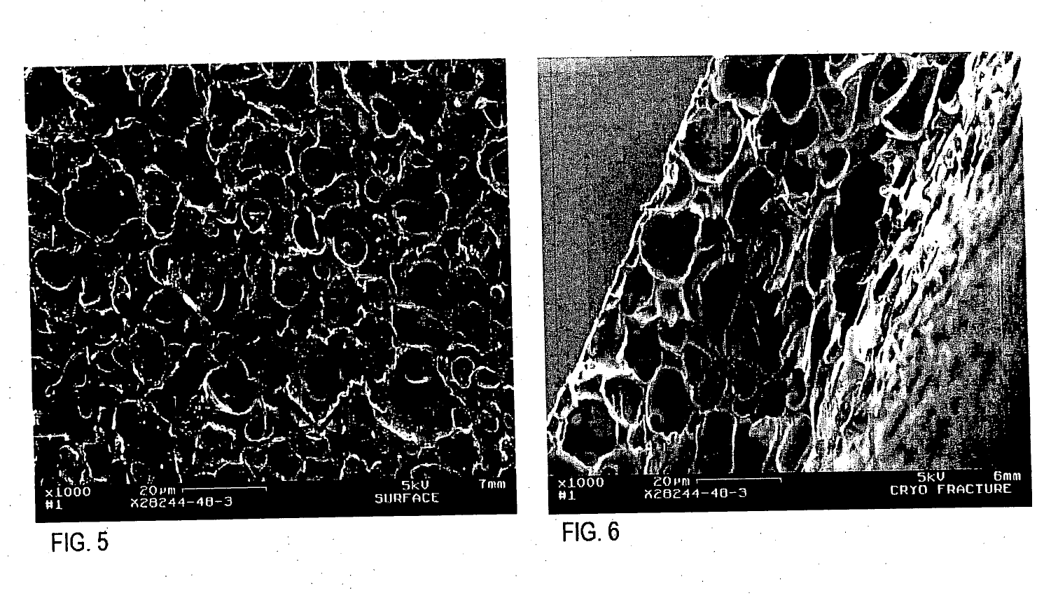

From the above data, the majority of extractable glycerol has been removed within the 24 hour extraction period. Referring to FIGS. 3-6, electron photomicrographs show the films having a highly desirable reticulated morphology. Such morphology serves to increase the surface area of the fiber, and thereby exposing dissolved nutrients in the fiber for extraction by digestive processes.

### EXAMPLES 4 and 5

Confectioners' powdered sugar with particle size of 1-10 microns was added to the cellulose diacetate:acetone solution of Example 1 at a weight ratio of 1:1 powdered sugar:cellulose diacetate (weight basis). The sugar was insoluble in the acetone solution, but was thoroughly agitated until visibly homogeneous.

In a vial, an amount of powdered sugar was added to a 10.0.0 gram aliquot of 10.0% cellulose diacetate in acetone to bring the mixture to the specified cellulose diacetate:sugar ratio shown in Table 2 below. The vial was sealed and then agitated until the sample was homogeneous. A 20 micron film was drawn down on a glass plate and set aside to dry. The film was allowed to dry overnight. A piece of the film was then removed, weighed, and placed in another vial containing sodium phosphate/sodium hydroxide solution buffered to pH 7.3. The vial was gently agitated for 24 hours at approximately 25° C., after which time it was removed from the liquid, dried, and weighed. The extraction procedure was repeated once more. The extraction data are given in Table II below.

TABLE II

Example #

| | 4 | 5 |
|---|---|---|
| ratio CE:sugar | 1:1 | 1:2 |
| Dry film wt, initial | 0.299 g | 0.537 g |
| Dry film wt, after 1 extraction | 0.091 g | 0.068 g |
| Dry film wt, after 2 extractions | 0.084 g | 0.071 g |

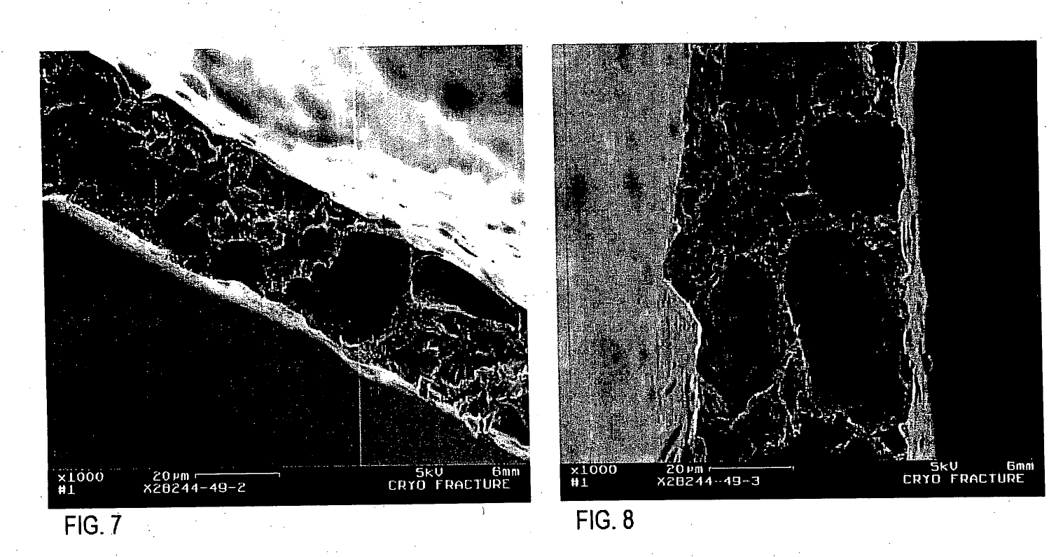

From the above data, the majority of extractable sugar has been removed within the 24 hour extraction period. Referring to FIGS. 7 and 8, electron photomicrographs show the films having a highly desirable reticulated morphology. Such morphology serves to increase the surface area of the fiber, and thereby exposing dissolved nutrients in the fiber for extraction by digestive processes.

### EXAMPLE 6

In a vial, 1.0 gram of water-soluble starch (EmCap 12633, available from Cargill) was added to a 10.0 gram aliquot of 10.0% cellulose diacetate in acetone to make at a weight ratio of 1:1 starch:cellulose diacetate. The starch was insoluble in the solution but was thoroughly agitated until visibly homogeneous. The vial was sealed and then agitated until the sample was visually homogeneous. A 20 micron film was drawn down on a glass plate and set aside to dry. The film was allowed to dry overnight. A piece of the film was then removed, weighed, and placed in another vial containing sodium phosphate/sodium hydroxide solution buffered to pH 7.3. The vial was gently agitated for 24 hours at approximately 25° C., after which time it was removed from the liquid, dried, and weighed. The extraction procedure was repeated again. The extraction data are given in Table III below.

TABLE III

| | Example # |
|---|---|
| | 6 |
| Dry film wt, initial | 0.414 g |
| Dry film wt, after 1 extraction | 0.185 g |
| Dry film wt, after 2 extractions | 0.113 g |

From the above data, the majority of extractable sugar has been removed within the 24 hour extraction period. The film had a reticulated morphology.

### EXAMPLES 7-9

In a vial, an amount of propylene glycol was added to a 10.0.0 gram aliquot of 10.0% cellulose diacetate in acetone to bring the mixture to the specified propylene glycol:cellulose diacetate ratio shown in Table IV below. The vial was sealed and then agitated until the sample was homogeneous. A 20 micron film was drawn down on a glass plate and set aside to dry. The film was allowed to dry overnight. A piece of the film was then removed, weighed, and placed in another vial containing sodium phosphate/sodium hydroxide solution buffered to pH 7.3. The vial was gently agitated for 24 hours at approximately 25° C., after which time it was removed from the liquid, dried, and weighed. The extraction procedure was repeated again. The extraction data are given in Table IV below.

TABLE IV

| | Example # | | |
|---|---|---|---|
| | 7 | 8 | 9 |
| Ratio Propylene glycol:CA | 2:1 | 3:1 | 5:1 |
| Dry film wt, initial | 0.129 g | 0.148 g | 0.644 g |
| Dry film wt, after 1 extraction | 0.091 g | 0.088 g | 0.068 g |
| Dry film wt, after 2 extractions | 0.084 g | 0.082 g | 0.068 g |

From the above data, the majority of extractable propylene glycol has been removed within the 24-hour extraction period. The film had a reticulated morphology but the degree of reticulation was less than that achieved using either glycerol, sugar, or starch.

### EXAMPLE 10

In a vial, 1.0 gram of citric acid was added to a 10.0 gram aliquot of 10.0% cellulose diacetate in acetone. The vial was sealed and then agitated until the sample was homogeneous. A 20 micron film was drawn down on a glass plate and set aside to dry. The film was allowed to dry overnight. A piece of the film was then removed, weighed, and placed in another vial containing sodium phosphate/sodium hydroxide solution buffered to pH 7.3. The vial was gently agitated for 24 hours at approximately 25° C., after which time it was removed from the liquid, dried, and weighed. The extraction procedure was repeated again. The extraction data are given in Table V below.

TABLE V

| | Example # |
|---|---|
| | 10 |
| Dry film wt, initial | 0.414 g |
| Dry film wt, after 1 extraction | 0.185 g |
| Dry film wt, after 2 extractions | 0.113 g |

From the above data, the majority of extractable citric acid has been removed within the 24-hour extraction period. The film had a reticulated morphology but the degree of reticulation was less than that achieved using either glycerol, sugar, or starch.

### EXAMPLES 11-15

Spun fibers were prepared. A fiber dope mix was prepared using the amount of acetone and cellulose diacetate, by weight, presented in Table VI below and mixing as per usual solvating procedures prior to adding glycerin. Standard procedures to prevent acetone loss were followed. Fifteen minutes after the glycerin was added, a solids test was performed. No water was intentionally added, but prior to spinning the fiber the water content in the fiber dope was measured.

TABLE VI

| Example | | | | glycerin % | % Water in |
|---|---|---|---|---|---|
| No. | Ester | acetone | glycerin | of solids | dope |
| 11 | 30 | 66 | 4 | 12 | 1.33 |
| 12 | 25 | 68 | 7 | 22 | 1.11 |
| 13 | 28 | 64 | 8 | 22 | 0.87 |
| 14 | 23 | 66 | 11 | 32 | 1.47 |
| 15 | 33 | 60 | 7 | 18 | 1.21 |

Samples from Examples 11, 12 and 14 were selected for extraction. The samples were placed in a glass jar, approximately 100 grams of simulated intestinal fluid (pH 6.8) was added and the jars were sealed. The jars were rolled at room temperature for one day. The samples were rinsed with deionized water, dried (vacuum filter for 20 minutes) and weighed. The samples were then put back into new fluid and extracted for one more day. The results are presented in Table VII below.

TABLE VII

| Example | Starting | 1 Day extraction | 2 Day extraction |
|---|---|---|---|
| No. | weight (g) | weight (g) | weight (g) |
| 11 | 0.51 | 0.44 | 0.45 |
| 12 | 0.48 | 0.38 | 0.36 |
| 14 | 0.50 | 0.37 | 0.33 |

The weight loss is consistent with all of the glycerin being extracted.

### EXAMPLES 16-18

Samples from Examples 11, 12 and 14 were selected for extraction. The samples were placed in a glass jar and approximately 100 grams of simulated stomach fluid (pH 1.2) was added, the jars were sealed and rolled for two hours at room temperature. The samples were rinsed thoroughly with deionized water, dried (vacuum filter for 20-40 minutes) and weighed. The weights were in the 10 gram range indicating that the samples at the very least were not dry. They were then put into a 45° C. oven overnight. The results appear in Table VIII below.

TABLE VIII

| Example | Sample | Starting weight | 2 Hr extraction | % glycerin |
|---|---|---|---|---|
| No. | No. | (g) | weight (g) | remaining |
| 16 | 11 | 8.23 | 7.46 | 0.1 |
| 17 | 12 | 8.54 | 6.95 | 0.2 |
| 18 | 14 | 8.44 | 5.91 | NA |

The weight loss recorded shows that the majority of the glycerin is extracted. The analytical analysis shows that essentially all of the glycerin was extracted over a two hour time period.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

What is claimed is:

1. A composition comprising:
a. a biocompatible primary polymer matrix forming a continuous phase of said composition; and
b. an active agent forming a substantially discontinuous phase of said composition,
wherein said biocompatible primary polymeric matrix has a glass transition temperature of less than about 100° C. and is impermeable to said active agent.

2. The composition of claim 1 wherein said primary biocompatible polymeric matrix is selected from the group consisting of cellulose acetate, cellulose propionate, and cellulose butyrate.

3. The composition of claim 2 wherein said biocompatible polymeric matrix comprises from about 5 weight % to 100 weight % of said primary polymer and up to about 95 weight % of a secondary polymer, wherein the weight % is based on the total weight of the polymer matrix.

4. The composition of claim 3 wherein said biocompatible polymeric matrix comprises from about 25 weight % to about 90 weight % of said primary polymer and from about 75 weight % to about 10 weight % of said secondary polymer, wherein the weight % is based on the total weight of polymer in the matrix.

5. The composition of claim 3 wherein said biocompatible polymeric matrix comprises greater than about 50 weight % of the primary polymer with the remainder of the polymer matrix comprising the secondary biocompatible polymer.

6. The composition of claim 1 wherein the amount of active agent in said composition is from about 0.01 weight % to about 70 weight % based on the total weight of the polymeric matrix and active agent.

7. The composition of claim 1 wherein the amount of active agent in said composition is from about 1.0 weight % to about 35 weight % based on the total weight of the polymeric matrix and active agent.

8. The composition of claim 1 wherein the amount of active agent in said composition is from about 1.0 weight % to about 25 weight % based on the total weight of the polymeric matrix and active agent.

9. The composition of claim 1 wherein when said active agent is selected from the group consisting of immunosuppressants, water and oil soluble vitamins, antioxidants, anesthetics, chemotherapeutic agents, steroids (including retinoids), hormones, antibiotics, anti-virals, anti-fungals, anti-tumor agents antiproliferatives, antihistamines, anticoagulants, antiphotoaging agents, melanotropic peptides, nonsteroidal and steroidal anti-inflammatory compounds, antipsychotics, and radiation absorbers, including UV-absorbers, lipids, polypeptides, proteins and mixtures thereof.

10. The composition of claim 1 further comprising a solubility enhancing agent selected from a water soluble tocopherol, wherein said water soluble tocopherol is prepared by esterifying a tocopheryl acid ester with polyethylene glycol, and wherein said polyethylene glycol has a molecular weight in the range of about 200 to 20,000.

11. The composition of claim 10 wherein said polyethylene glycol has a molecular weight in the range of about 400 to about 1000.

12. The composition of claim 10 wherein said solubility enhancing agent is Vitamin E polyethylene glycol 1000 succinate.

13. The composition of claim 12 wherein an amount of solubility enhancing agent incorporated into said composition is from about 0.02 to about 10 weight %, based on the total weight of said active agent and solubility enhancing agent.

14. A polymeric article comprising:
a. from about 5 weight % to 100 weight % of a biocompatible primary polymer matrix forming a continuous phase of said article wherein said primary polymer matrix is selected from the group consisting of cellulose acetate, cellulose propionate, and cellulose butyrate; and
b. from about 0.01 weight % to about 70 weight % an active agent forming a substantially discontinuous phase of said article,
wherein said biocompatible primary polymeric matrix has a glass transition temperature of less than about 100° C. and is impermeable to said active agent and wherein said weight % is based on the total weight of the polymeric matrix and active agent.

15. The article of claim 14 wherein said article is a fiber having a denier of from about 1 to about 5000.

16. The article of claim 14 wherein said article is a film.

17. The article of claim 14 wherein said biocompatible polymeric matrix further comprises up to about 95 weight % of a secondary polymer, wherein the weight % is based on the total weight of the polymer matrix.

18. The article of claim 14 wherein said biocompatible polymeric matrix comprises from about 25 weight % to about 90 weight % of said primary polymer and from about 75 weight % to about 10 weight % of said secondary polymer, wherein the weight % is based on the total weight of polymer in the matrix.

19. The article of claim 14 wherein the amount of active agent is from about 1.0 weight % to about 10 weight % based on the total weight of the polymeric matrix and active agent.

20. The article of claim 14 further comprising from about 0.02 to about 10 weight % of a solubility enhancing agent selected from a water soluble tocopherol, wherein said water soluble tocopherol is prepared by esterifying a tocopheryl acid ester with polyethylene glycol, and wherein said polyethylene glycol has a molecular weight in the range of about 400 to about 1000, and wherein the weight % is based on the total weight of said active agent and said solubility enhancing agent.

21. A method for preparing an article of claim 14 comprising:
a. providing a polymer blend comprising a polymer matrix having from 99.9 weight % to about 30 weight % of a biocompatible polymeric material and from about 0.1 to about 70 weight % of an active agent, based on the total weight of said polymer matrix and active agent; and
b. extruding said polymer blend to form said article, wherein said biocompatible polymeric matrix has a glass transition temperature of less than about 100° C., and said biocompatible polymeric material is impermeable to said active agent.

22. The method of claim 21 wherein said blend is a dry blend.

23. The method of claim 21 wherein said blend is a solubilized blend.

24. The method of claim 21 further comprising adding from about 1:1 to about 5:1, based on a dry weight basis of the polymer matrix, of a morphology promoting agent to said blend prior to the extruding step.
